(19) United States
(12) Patent Application Publication
(10) Pub. No.: US 2023/0346362 A1
(43) Pub. Date: Nov. 2, 2023

(54) BLADE ALIGNMENT DEVICE

(71) Applicant: MiRus LLC, 29827332, GA (US)

(72) Inventor: Jared Michael White, Acworth, GA (US)

(21) Appl. No.: 18/219,766

(22) Filed: Jul. 10, 2023

Related U.S. Application Data
(63) Continuation-in-part of application No. 29/827,332, filed on Feb. 18, 2022.
(60) Provisional application No. 63/359,439, filed on Jul. 8, 2022.

(51) Int. Cl. A61B 17/02

(52) U.S. Cl. CPC A61B 17/02

(57) ABSTRACT

A blade alignment device that engages one or more blades of a retractor device to maintain the alignment of one or more blades of the retractor device during insertion of the retractor device in a patient.

The present disclosure is a continuation-in-part of U.S. Design Patent Application Ser. No. 29/827,332 filed Feb. 18, 2022, which is fully incorporated herein by reference.

The present disclosure claims priority on U.S. Provisional Patent Application Ser. No. 63/359,439 filed Jul. 8, 2022, which is fully incorporated herein by reference.

The present disclosure is directed to medical devices, particularly to medical devices for use with surgical devices, more particularly to blade alignment device that engages one or more blades of a retractor device to maintain the alignment of one or more blades of the retractor device during insertion of the retractor device in a patient, and even more particularly to blade alignment device that temporarily engages one or more blades of a retractor device to maintain the alignment of one or more blades of the retractor device during insertion of the retractor device in a spinal region of a patient during spinal surgery, and which blade alignment device is removable from the retractor device after the retractor device has been positioned in a desired location during spinal surgery.

### BACKGROUND OF THE DISCLOSURE

During spinal surgery, retractor systems are commonly used to facilitate in the insertion of implants in the spinal region of a patient. Generally, a patient's skin is cut and the retractor system is inserted through the cut in the skin and then further inserted to the site wherein the implant is to be inserted. Thereafter, the blades of the retractor system are opened to clear a region about the surgical site so that the surgeon can insert an initial dilator, implant, or the like though the retractor system and to the surgical site.

When initially inserting the retractor system into the patient, one or more of the blades at the distal end of the retractor system can become misaligned due to contact with bone and/or tissue as the retractor system is inserted into the patient. Such misalignment of one or more blades of the retractor system can cause damage to the retractor system, namely, a) the bending of one or more blades, b) the twisting of one or more blades, and/or c) damage to the blade opening and/or blade closing mechanism. The blade misalignment of the retractor system during insertion into the patient can also result in improper insertion of the retractor system at the desired surgical site location, and/or undesired injury or damage to bone and/or tissue as the retractor system is inserted and/or removed from the patient.

In view of the current state of retractor systems, there is a need for a blade alignment device that can be easily be used with a retractor system to facilitate in maintaining the alignment of one or more of the blades of the retractor device during insertion of the retractor device in a patient.

### SUMMARY OF THE DISCLOSURE

The present disclosure is directed to a blade alignment device that engages one or more blades of a retractor device to maintain the alignment of one or more blades of the retractor device during insertion of the retractor device in a patient. In one non-limiting embodiment, the blade alignment device is configured to temporarily engage one or more blades of a retractor device to maintain the alignment of one or more blades of the retractor device during insertion of the retractor device in a spinal region of a patient during spinal surgery, and wherein the blade alignment device is also configured to be removable from the retractor device after the retractor device has been positioned in a desired location during spinal surgery. In one non-limiting embodiment, the retractor device includes a plurality of blades (e.g., 2-6 blades that are in a fixed position relative to the body of the retractor device. The blades of the retractor device are generally connected to movable arms of the retractor device. Each of blades include side walls that can optionally be arcuate shaped. Positioned at the end of each of the side walls is an outwardly extending wall flange that can run partially or fully along the longitudinal length of the blade.

In one non-limiting aspect of the disclosure, blade alignment device in accordance with the present disclosure includes an elongated body, a first end portion, a second end portion, and a blade engagement arrangement that is located at the second end portion. In one non-limiting embodiment, the longitudinal length of blade alignment device is 50-150% (and all values and ranges therebetween) the longitudinal length of the blades or the longitudinal length of the movable arms of the retractor device. In another non-limiting embodiment, the longitudinal length of blade alignment device is selected so that at least a portion of first end portion of blade alignment device extends rearwardly from blades and/or the movable arms of the retractor device when blade alignment device is fully inserted into retractor device and while the blades are in the closed position.

In another and/or alternative non-limiting aspect of the disclosure, the shape of elongated body between all or a majority of the first end portion and second end portion optionally has a generally constant cross-sectional shape and/or cross-sectional area. In one non-limiting embodiment, the shape of elongated body between all or a majority of the first end portion and second end portion is generally cylindrical shaped and has a constant circular diameter. In another non-limiting embodiment, the elongated body can be solid, or include one or more internal cavities along the longitudinal length of elongated body. In one non-limiting configuration, the elongated body includes an internal cavity along a portion or all of the longitudinal length of the elongated body and the internal cavity has a constant cross-sectional shape (e.g., circular cross-sectional shape, etc.) along a portion or all of the longitudinal length of internal cavity.

In another and/or alternative non-limiting aspect of the disclosure, the first end portion of blade alignment device optionally includes one or more gripping members. The one or more gripping members can be used to facilitate in the handling of blade alignment device (e.g., facilitate in the insertion and/or removal of blade alignment device into/from the retractor device, gripping of blade alignment device, etc.). The number and/or shape of gripping members is non-limiting. In one non-limiting embodiment, the gripping member includes one or more grooves (e.g., 2-10 grooves and all values and ranges therebetween) that partially or fully encircle the elongated body. Generally, gripping member, when used is located on no more than 30% (e.g., 1-30% and all values and ranges therebetween) of the longitudinal length of blade alignment device. The gripping member, when used, can be spaced from or extend to the end of the elongated body.

In another and/or alternative non-limiting aspect of the disclosure, the second end portion of blade alignment device includes a blade engagement arrangement. The blade engagement arrangement includes one or more retention structures that are configured to at least partially surround and/or engage a portion of one or more wall flanges of one or more of the blades of the retractor device to facilitate in maintaining the alignment and/or position of two or more blades of the retractor device relative to one another when two or more of the blades of the retractor device are in the closed position. In one non-limiting embodiment, a plurality of retention structures on the blade engagement arrangement are configured to at least partially surround and/or engage a portion of one or more wall flange of one or more of the blades of the retractor device to facilitate in maintaining the alignment and/or position of one or more or all of the blades of the of the retractor device relative to one another when the blades are in the closed position. The shape, size, and configuration of one or more retention structures of the blade engagement arrangement are non-limiting. In another non-limiting embodiment, the blade engagement arrangement includes the same number of retention structures as the number of blades of the tractor device.

In another and/or alternative non-limiting aspect of the disclosure, each retention structure of the blade engagement arrangement is configured to at least partially entrap or surround two wall flanges of the blades of the retractor device when the blades are in the closed position, wherein one wall flange is located on one blade and the other wall flange is located on another adjacently positioned blade. In one non-limiting embodiment, one or more or all of the retention structures has a generally C-shaped configuration; however, it will be appreciated that one or more of retention structures can have other shapes. In one non-limiting arrangement, the base of the retention structure has a width that is greater than the width at the top of the retention structure. In another non-limiting embodiment, one or more or all of the retention structures include first and second side walls wherein a) the first and second sides walls have the same length and/or shape, b) the first and second side walls angle toward one another from the base to the top of the retention structure, c) the first and second side walls have the same angle relative to the base of the retention structure, d) the top edges of the first and second sides walls are spaced from one another, e) the width of the base of the retention structure is greater than a longitudinal length of the first side wall or the second side wall, and/or f) a there is a curved transition between the bottom portion of the first and/or second side walls and the base of the retention structure. In another non-limiting embodiment, each of retention structures has the same size and configuration; however, this is not required. In another non-limiting embodiment, the adjacently positioned retention structures are equally spaced from one another about the outer perimeter of the elongated body.

In another and/or alternative non-limiting aspect of the disclosure, the blade engagement arrangement is spaced inwardly from the end of blade alignment device; however, this is not required. In one non-limiting arrangement, the blade engagement arrangement is spaced inwardly from the end of blade alignment device a distance of 0-20% (and all values and ranges therebetween) the longitudinal length of blade alignment device.

In another and/or alternative non-limiting aspect of the disclosure, the longitudinal length of the blade engagement arrangement is generally less than the longitudinal length of blade alignment device. In one non-limiting embodiment, the longitudinal length of the blade engagement arrangement is 2-40% (and all values and ranges therebetween) the longitudinal length of blade alignment device.

In another and/or alternative non-limiting aspect of the disclosure, the blade engagement arrangement includes a plurality of flat sides that extend between the retention structures. In one non-limiting embodiment, the number of flat sides is the same as the number of retention structures; however, this is not required. In another non-limiting embodiment, the longitudinal length of two or more or all of the flat sides is the same length; however, this is not required. In another non-limiting embodiment, a majority (e.g., 50.001-99.99% and all values and ranges therebetween) or all of the longitudinal length of the flat side edges angle toward adjacently positioned flat side edges. In another non-limiting embodiment, the first and/or second end portions of one or more or all of the flat side edges is non-parallel to the plane of the mid-region of the flat side edges. In another non-limiting embodiment, each of the first and second end portions has a longitudinal length that is 1-40% (and all values and ranges therebetween) (e.g., 1-15%, etc.) the longitudinal length of the flat side edge.

In one non-limiting object of the present disclosure is the provision of a blade alignment device adapted for use with a retractor device that is configured to engage one or more blades of a retractor device to maintain the alignment of one or more blades of the retractor device during insertion of the retractor device in a patient.

In another non-limiting object of the present disclosure is the provision of a blade alignment device that is configured to temporarily engage one or more blades of a retractor device to maintain the alignment of one or more blades of the retractor device during insertion of the retractor device in a spinal region of a patient during spinal surgery, and wherein the blade alignment device is also configured to be removable from the retractor device after the retractor device has been positioned in a desired location during spinal surgery.

In another non-limiting object of the present disclosure is the provision of a blade alignment device that is configured to temporarily engage one or more blades of a retractor device to maintain the alignment of one or more blades of the retractor device during insertion of the retractor device in a spinal region of a patient during spinal surgery, and wherein the blade alignment device is also configured to be removable from the retractor device after the retractor device has been positioned in a desired location during spinal surgery, and wherein blade alignment device includes an elongated body and a blade engagement arrangement; the elongated body includes first and second ends, a first end portion, and a second end portion; the blade engagement arrangement is located at the second end portion; the blade engagement arrangement includes a first retention structure; the first retention structure is configured to at least partially surround and/or engage a portion of a plurality of blades on the retractor device to facilitate in maintaining alignment and/or position of the plurality of blades relative to one another when the plurality blades are in a closed position.

In another non-limiting object of the present disclosure is the provision of a blade alignment device that is configured to temporarily engage one or more blades of a retractor device to maintain the alignment of one or more blades of the retractor device during insertion of the retractor device in a spinal region of a patient during spinal surgery, and wherein the blade alignment device is also configured to be removable from the retractor device after the retractor device has been positioned in a desired location during spinal surgery, and wherein blade alignment device includes an elongated body and a blade engagement arrangement; the elongated body includes first and second ends, a first end portion, and a second end portion; the blade engagement arrangement is located at the second end portion; the blade engagement arrangement includes a first retention structure; the first retention structure is configured to at least partially surround and/or engage a portion of first and second blades on the retractor device that are located adjacent to one another to facilitate in maintaining alignment and/or position of the first and second blades relative to one another when the first and second blades are in a closed position.

In another non-limiting object of the present disclosure is the provision of a blade alignment device that is configured to temporarily engage one or more blades of a retractor device to maintain the alignment of one or more blades of the retractor device during insertion of the retractor device in a spinal region of a patient during spinal surgery, and wherein the blade alignment device is also configured to be removable from the retractor device after the retractor device has been positioned in a desired location during spinal surgery, and wherein blade alignment device includes an elongated body and a blade engagement arrangement; the elongated body includes first and second ends, a first end portion, and a second end portion; the blade engagement arrangement is located at the second end portion; the blade engagement arrangement includes first and second retention structures; the first retention structure is configured to at least partially surround and/or engage a portion of first and second blades on the retractor device that are located adjacent to one another to facilitate in maintaining alignment and/or position of the first and second blades relative to one another when the first and second blades are in a closed position; the second retention structure is configured to at least partially surround and/or engage a portion of second and third blades on the retractor device that are located adjacent to one another to facilitate in maintaining alignment and/or position of the second and third blades relative to one another when the second and third blades are in a closed position.

In another non-limiting object of the present disclosure is the provision of a blade alignment device that is configured to temporarily engage one or more blades of a retractor device to maintain the alignment of one or more blades of the retractor device during insertion of the retractor device in a spinal region of a patient during spinal surgery, and wherein the blade alignment device is also configured to be removable from the retractor device after the retractor device has been positioned in a desired location during spinal surgery, and wherein blade alignment device includes an elongated body and a blade engagement arrangement; the elongated body includes first and second ends, a first end portion, and a second end portion; the blade engagement arrangement is located at the second end portion; the blade engagement arrangement includes first, second and third retention structures; the first retention structure is configured to at least partially surround and/or engage a portion of the first and second blades on the retractor device that are located adjacent to one another to facilitate in maintaining alignment and/or position of the first and second blades relative to one another when the first and second blades are in a closed position; the second retention structure is configured to at least partially surround and/or engage a portion of the second and third blades on the retractor device that are located adjacent to one another to facilitate in maintaining alignment and/or position of the second and third blades relative to one another when the second and third blades are in a closed position; the third retention structure is configured to at least partially surround and/or engage a portion of the first and third blades on the retractor device that are located adjacent to one another to facilitate in maintaining alignment and/or position of the first and third blades relative to one another when the first and third blades are in a closed position.

In another non-limiting object of the present disclosure is the provision of a blade alignment device that includes an elongated body that has a generally cylindrical shape between the first end portion and said second end portion.

In another non-limiting object of the present disclosure is the provision of a blade alignment device that includes an elongated body that has an internal cavity that runs along 50-100% a longitudinal length of the elongated body.

In another non-limiting object of the present disclosure is the provision of a blade alignment device that includes a gripping member that is positioned on the first end portion of the elongated body; the gripping member includes one or more gripping features; the gripping member is optionally located on no more than 30% of said longitudinal length of the elongated body.

In another non-limiting object of the present disclosure is the provision of a blade alignment device that the one or more retention structures include first and second side walls that are configured to at least partially entrap or surround at least a portion of a plurality of blades of the retractor device when the blades are in the closed position.

In another non-limiting object of the present disclosure is the provision of a blade alignment device wherein the first and second side walls of the one or more retention structures angle inwardly toward one another; the first and second walls of one or more of the retention structures optionally extend outwardly from an outer perimeter of the elongated body.

In another non-limiting object of the present disclosure is the provision of a blade alignment device wherein one or more of the retention structures has a generally C-shaped configuration.

In another non-limiting object of the present disclosure is the provision of a blade alignment device wherein the blade engagement arrangement is spaced inwardly from the second end of the elongated body; the blade engagement arrangement is optionally spaced inwardly from said second end of the elongated body a distance of 0.5-20% the longitudinal length of the elongated body.

In another non-limiting object of the present disclosure is the provision of a blade alignment device that includes a blade engagement arrangement that has a longitudinal length that is less than the longitudinal length of the elongated body; the longitudinal length of said blade engagement arrangement is optionally 2-40% the longitudinal length of the elongated body.

In another non-limiting object of the present disclosure is the provision of a method for using a blade alignment device to at least partially maintain alignment and/or positioning of a plurality of retractor device blades relative to one another when the plurality of retractor device blades are in a closed position; the method comprises a) providing a retractor device; the retractor device comprises a body, first and second arms, a first blade connected to the first arm, and a second blade connected to the second arm; the first and second blades are movable between a closed position and an open position; the first and second blades at least partially form a blade cavity when the first and second blades are in the closed position; b) providing a blade alignment device as described above; the blade alignment device is adapted for use with the retractor device; c) at least partially inserting the blade alignment device in the blade cavity such that the first retention structure at least partially surrounds and/or engages a portion of the first and second blades to facilitate in maintaining alignment and/or position of the first and second blades relative to one another when the first and second blades are in the closed position.

In another non-limiting object of the present disclosure is the provision of a method for using a blade alignment device to at least partially maintain alignment and/or positioning of a plurality of retractor device blades relative to one another when the plurality of retractor device blades are in a closed position; and wherein each of the first and second blades includes first and second side walls that are optionally arcuate shaped, and wherein an end portion of each of the first and second side walls includes an outwardly extending wall flange; the first and second wall flanges run partially or fully along a longitudinal length of the first and second blades; and wherein the first retention structure is configured to at least partially surround, engage, and/or entrap a portion of the first side wall on the first blade and the second side wall of the second blade when the blade alignment device is at least partially inserted in the blade cavity.

In another non-limiting object of the present disclosure is the provision of a method for using a blade alignment device to at least partially maintain alignment and/or positioning of a plurality of retractor device blades relative to one another when the plurality of retractor device blades are in a closed position; and wherein the longitudinal length of the elongated body is 80-150% a longitudinal length of the first blade and/or the second blade.

In another non-limiting object of the present disclosure is the provision of a method for using a retractor device in a patient comprising a) providing a retractor device; the retractor device comprising a body, first and second arms, a first blade connected to said first arm, and a second blade connected to said second arm; the first and second blades are movable between a closed position and an open position; the first and second blades at least partially form a blade cavity when the first and second blades are in the closed position; b) positioning the first and second blades of the retractor device in the closed position; c) providing a blade alignment device as discussed above; said blade alignment device adapted for use with the retractor device; d) at least partially inserting the blade alignment device in the blade cavity such that the first retention structure at least partially surrounds and/or engages a portion of the first and second blades to facilitate in maintaining alignment and/or position of the first and second blades relative to one another when the first and second blades are in the closed position; e) inserting at least a portion of the first and second blades of the retractor device into the patient until a front end of the first and second blades is located at a desired location in the patient; f) removing the blade alignment device from the retractor device; and, g) causing the first and second blades to move from the closed position to the open position while at least a portion of the first and second blades are located in the patient; and wherein the maintaining alignment and/or position of the first and second blades relative to one another by the blade alignment device optionally inhibits or prevents a) the distal end of the blades of the retractor device from becoming misaligned due to contact with bone and/or tissue as the blades of the retractor device are inserted into the patient, b) damage to the blades of the retractor device and/or other components of the retractor device due to one or more bent blades, the twisting of one or more blades, damage to the blade opening and closing mechanism, etc., c) improper insertion of the retractor device into the patient, and/or d) undesired injury or damage to bone and/or tissue as the blades of the retractor device are inserted into the patient.

### BRIEF DESCRIPTION OF THE DRAWINGS

The above and other features and advantages of the disclosure will become apparent from a consideration of the subsequent detailed description presented in connection with the accompanying drawings in which:

FIG. 1 is a side elevation view of a blade alignment device in accordance with the new design;

FIG. 2 is a front plan view of the blade alignment device of FIG. 1;

FIG. 3 is a back plan view of the blade alignment device of FIG. 1;

FIG. 4 is a top plan view of the blade alignment device of FIG. 1;

FIG. 5 is a bottom plan view of the blade alignment device of FIG. 1;

FIG. 6 is a side plan view of the blade alignment device of FIG. 1;

FIG. 7 is a cross-sectional view along a longitudinal axis of a retractor device in the closed position that includes the blade alignment device of FIG. 1;

FIGS. 8-11 are pictures of a retractor device in the closed position that includes the blade alignment device of FIG. 1; and,

FIG. 12 is a picture of the retractor device in the open position without the blade alignment device.

### DETAILED DESCRIPTION OF VARIOUS NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

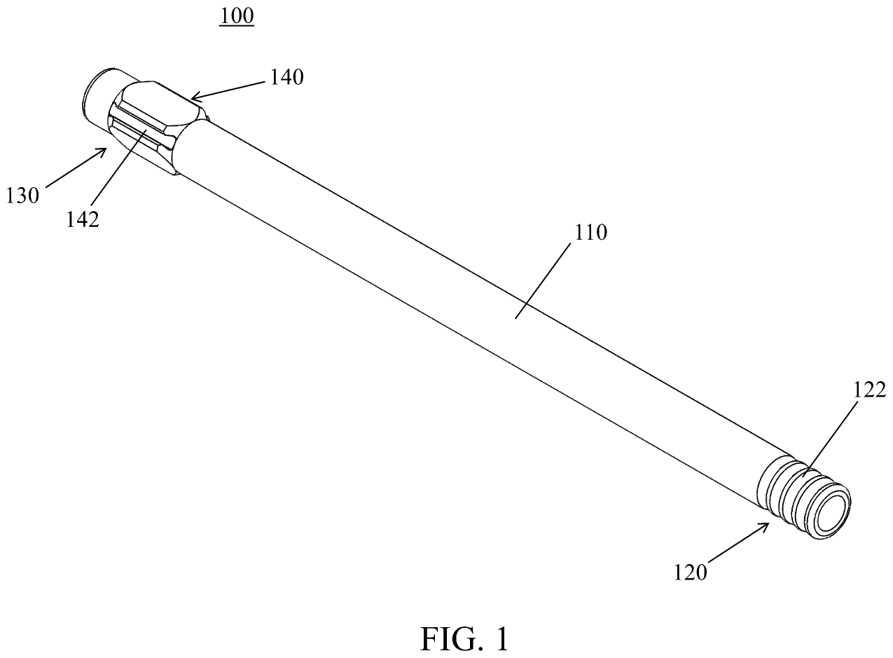

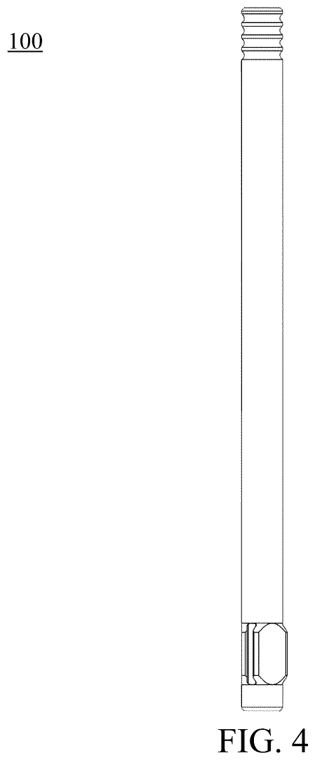

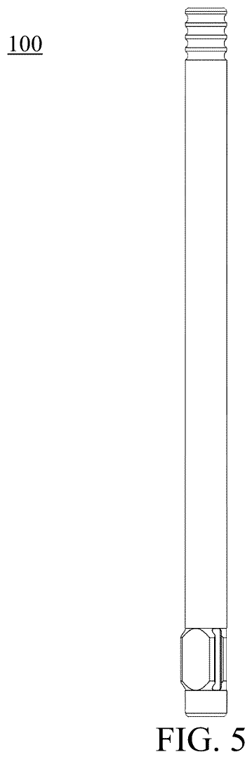

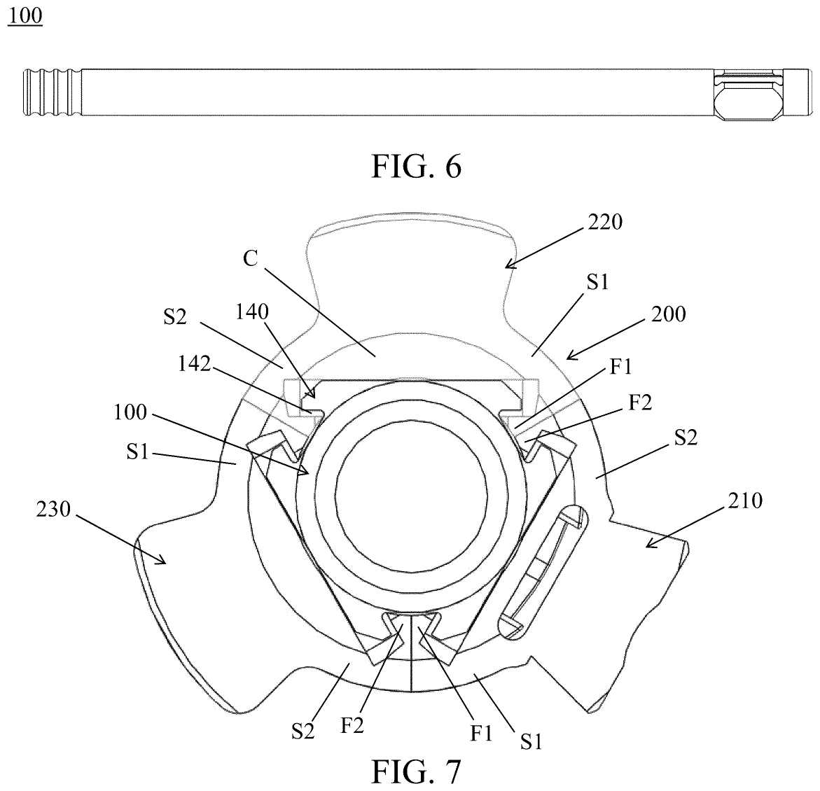

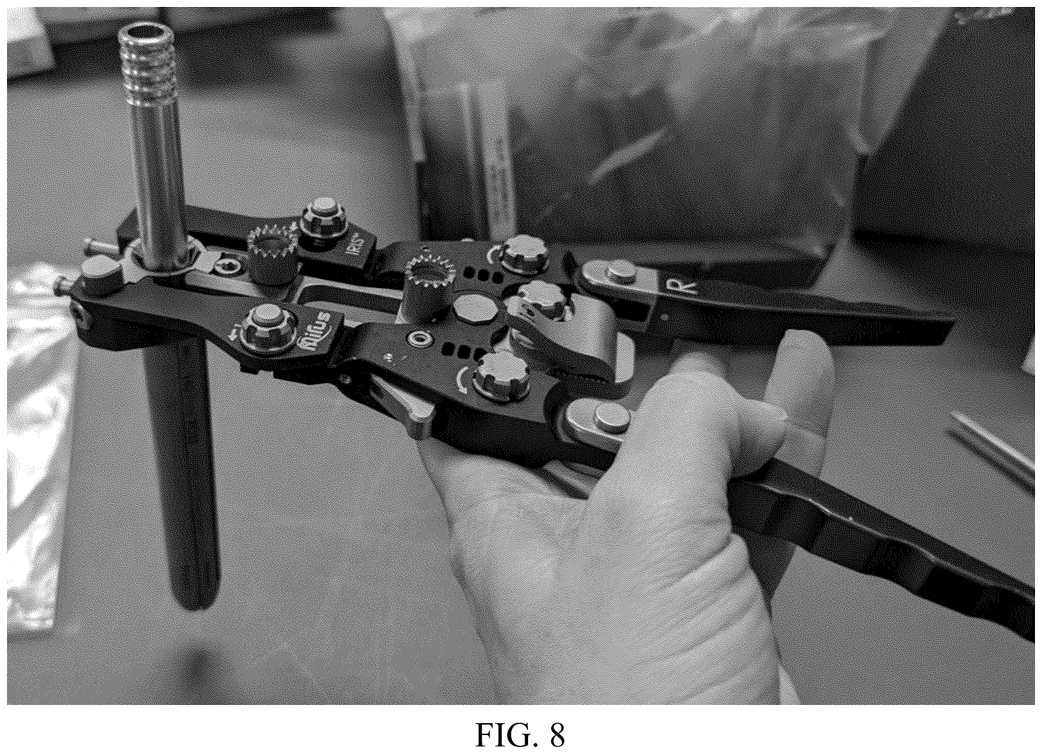

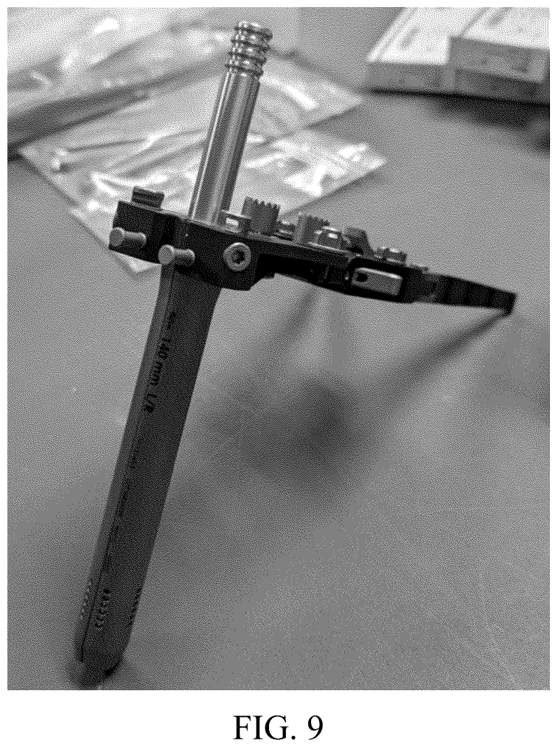

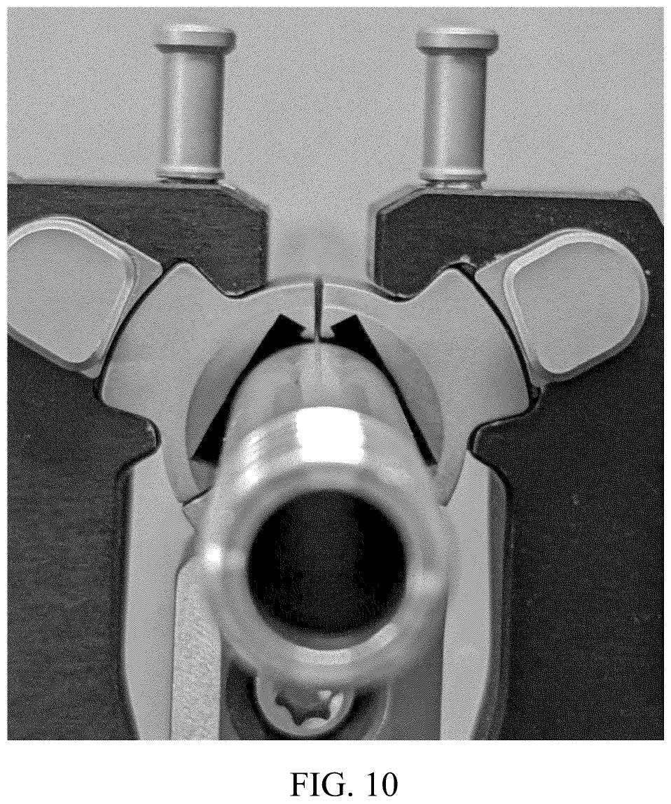

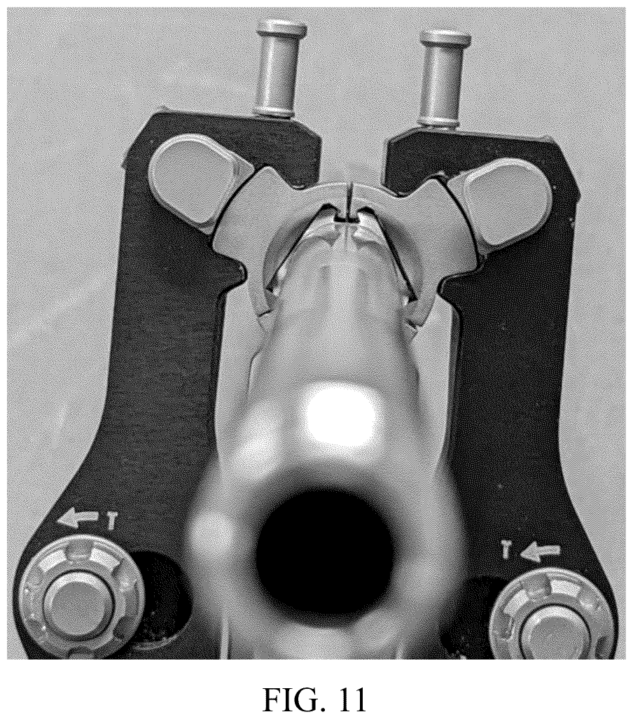

Referring now to the drawings, FIGS. 1-11 illustrate a non-limiting configuration of a blade alignment device 100 adapted for use with a retractor device 200 in accordance the present disclosure. FIG. 7-11 illustrate blade alignment device 100 of FIGS. 1-6 used in conjunction with a retractor device 200. The blade alignment device 100 generally includes one or more materials that impart the desired properties to the blade alignment device 100 for operation and to also withstand the manufacturing processes that are needed to produce the blade alignment device 100. These manufacturing processes can include, but are not limited to, laser cutting, etching, annealing, drawing, pilgering, electroplating, electro-polishing, machining, plasma coating, 3D printed coatings, 3D printing, chemical vapor deposition, chemical polishing, cleaning, pickling, ion beam deposition or implantation, sputter coating, vacuum deposition, etc. In one non-limiting embodiment, at least a portion or all of the blade alignment device 100 is formed by a 3D printing process. In another non-limiting embodiment, the blade alignment device 100 is partially or fully formed of a metal material (e.g., stainless steel, titanium, Co—Cr alloy, TiAlV alloy, molybdenum alloy, rhenium alloy, Mo—Re alloy, tungsten alloy, etc.).

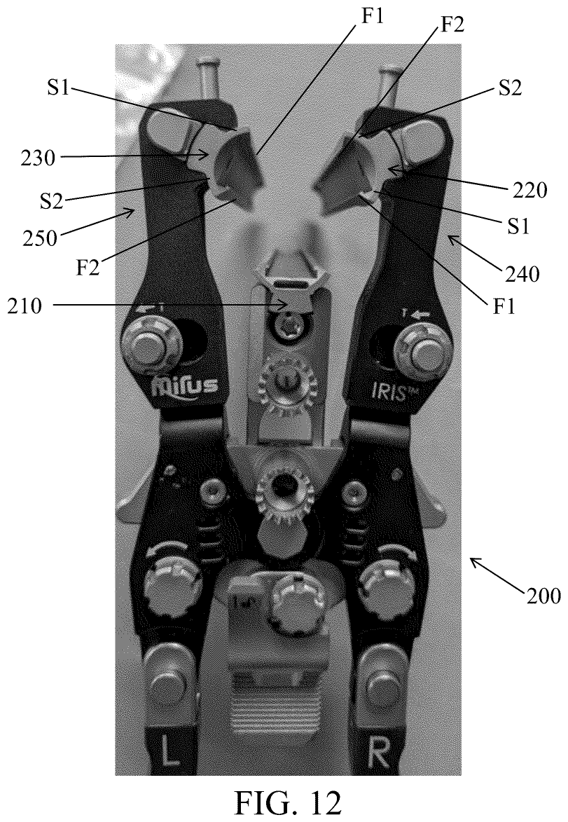

As best illustrated in FIG. 12, retractor device 200 includes three blades 210, 220, 230. Blade 210 is a lower blade that is fixed in position relative to the body 202 of the retractor device. Blades 220, 230 are connected to movable arms 240, 250 respectively. Each of blades 210, 220, 230 include side walls S1 and S2 that can optionally be arcuate shaped. Positioned at the end of each of the side walls is an outwardly extending wall flange F1 and F2. Wall flange F1 and F2 can run partially or fully along the longitudinal length of one or more of blades 210, 220, 230. The operation of a retractor device is known on the art, thus will not be described in detail herein. Prior art retractor devices are illustrated in U.S. Pat. No. 11,134,936 and US 2016/0317137, which are both fully incorporated herein by reference.

Referring again to FIGS. 1-6, there is illustrated a non-limiting blade alignment device 100 in accordance with the present disclosure. Blade alignment device 100 includes an elongated body 110, a first end portion 120, a second end portion 130, and a blade engagement arrangement 140 located at second end portion 130. The length of the elongated body 110 is non-limiting. In one non-limiting arrangement, the longitudinal length of blade alignment device 100 is 50-150% (and all values and ranges therebetween) the longitudinal length of blades 210, 220, 230. In one non-limiting arrangement, the longitudinal length of blade alignment device 100 is selected so that at least a portion of first end portion 120 of blade alignment device 100 extends rearward from blades 210, 220, 230 when blade alignment device 100 is fully inserted into retractor device 200 while the blades are in the closed position; however, such length is not required. In one non-limiting arrangement, the longitudinal length of blade alignment device 100 is 101-150% the longitudinal length of blades 210, 220, 230.

The shape of elongated body 110 between first end portion 120 and second end portion 130 is illustrated as being generally cylindrical shaped and having a constant circular diameter; however, it can be appreciated that elongated body 110 can have other cross-sectional shapes along one or more portions of the longitudinal length of elongated body 110 (e.g., square cross-sectional shape, rectangular cross-sectional shape, triangular cross-sectional shape, oval or ovoid cross-sectional shape, polygonal cross-sectional shape, stadium cross-sectional shape, semi-circular cross-sectional shape, etc.). Also, it can be appreciated that the diameter of a circular cross-sectional shaped elongated body 110 can have two or more different diameters along one or more portions of the longitudinal length of elongated body 110, and/or can have two or more different cross-sectional shapes along one or more portions of the longitudinal length of elongated body 110.

Elongated body 110 can be solid, or include one or more internal cavities along the longitudinal length of elongated body 110. In one non-limiting configuration, elongated body 110 includes an internal cavity along 50-100% (and all values and ranges therebetween) of the longitudinal length of elongated body 110. The internal cavity (when formed) can have any cross-sectional shape (e.g., circular cross-sectional shape, square cross-sectional shape, rectangular cross-sectional shape, triangular cross-sectional shape, oval or ovoid cross-sectional shape, polygonal cross-sectional shape, stadium cross-sectional shape, semi-circular cross-sectional shape, etc.). The cross-sectional shape and/or cross-sectional size of the internal cavity can be constant or varied along the longitudinal length of the internal cavity. As illustrated in FIG. 7, the elongated body 110 includes an internal cavity having a constant circular cross-sectional shape along 100% of the longitudinal length of elongated body 110.

Referring again to FIGS. 1-6, first end portion 120 of blade alignment device 100 optionally includes one or more gripping members 122. The one or more gripping members 122 can be used to facilitate in the handling of blade alignment device 100 (e.g., facilitate in the insertion and/or removal of blade alignment device 100 into/from retractor device 200, gripping of blade alignment device 100, etc.). The number and/or shape of gripping members 122 is non-limiting. As illustrated in in FIG. 1, a gripping member 122 in the form of a plurality of grooves (e.g., 2-5 grooves) that fully encircle elongated body 110 is formed on a portion the first end portion 120 of blade alignment device 100. As can be appreciated, the number of grooves used to form one or more gripping members 122 is non-limiting (e.g., 1-10 grooves and all values and ranges therebetween). As can all be appreciated, one or more of the grooves is not required to fully encircle elongated body 110. As also can be appreciated, other or additional configurations of gripping member 122 can be used (e.g., ribs, slots, roughened regions, etc.). Generally, gripping member 122 is located on no more than 30% (e.g., 1-30% and all values and ranges therebetween) of the longitudinal length of blade alignment device 100. As illustrated in FIG. 1, gripping member 122 is located on no more than 10% of the longitudinal length of blade alignment device 100.

Referring again to FIGS. 1-6, second end portion 130 of blade alignment device 100 includes blade engagement arrangement 140. Blade engagement arrangement 140 includes one or more retention structures 142 that are configured to at least partially surround and/or engage a portion of one or more wall flange F1 and F2 of one or more blades 210, 220, 230 to facilitate in maintain the alignment and/or position of two or more blades 210, 220, 230 relative to one another when two or more of the blades 210, 220, 230 are in the closed position. In one non-limiting arrangement, a plurality of retention structures 142 are configured to at least partially surround and/or engage a portion of one or more wall flange F1 and F2 of blades 210, 220, 230 to facilitate in maintain the alignment and/or position of all of blades 210, 220, 230 relative to one another when blades 210, 220, 230 are in the closed position.

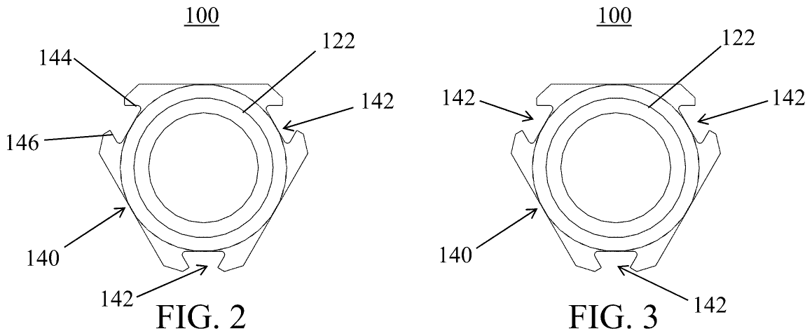

The shape, size, and configuration of one or more retention structures 142 of blade engagement arrangement 140 are non-limiting. Referring now to FIGS. 2, 3 and 7, blade engagement arrangement 140 includes three retention structures 142 wherein each retention structure is configured to at least partially entrap or surround two wall flanges on the blades of retractor device 200, wherein one wall flange is located on one blade and the other wall flange is located on another blade. Each of retention structures 142 has a generally C-shaped configuration; however, it will be appreciated that one or more of retention structures 142 can have other shapes (e.g., generally an L-shaped configuration, generally a J-shaped configuration, generally a V-shaped configuration, generally a U-shaped configuration, etc.). Each of retention structures 142 illustrated in FIGS. 2, 3 and 7 have the same size and configuration; however, this is not required. Each of retention structures 142 includes a cavity having one or more side walls 144, 146 that are configured to at least partially entrap or surround two wall flanges on the blades of the retractor device 200 when the blades are in the closed position. Side walls 144, 146 are illustrated as angling inwardly toward one another; however, this is not required. As illustrated in FIGS. 2, 3 and 7, side walls 144, 146 extend outwardly from the outer perimeter of body 110; however, this is not required. As can be appreciated, one or more retention structures 142 can have other shapes and configurations. As can be appreciated, the shape of retention structures 142 can be the same or different.

As illustrated in FIGS. 1 and 4-6, blade engagement arrangement 140 is spaced inwardly from the end of blade alignment device 100; however, this is not required. In one non-limiting arrangement, blade engagement arrangement 140 is spaced inwardly from the end of blade alignment device 100 a distance of 0-20% (and all values and ranges therebetween) the longitudinal length of blade alignment device 100. The longitudinal length of blade engagement arrangement 140 is generally less than the longitudinal length of blade alignment device 100; however, this is not required. In one non-limiting arrangement, the longitudinal length of blade engagement arrangement 140 is 2-40% (and all values and ranges therebetween) the longitudinal length of blade alignment device 100.

As illustrated in FIGS. 7-11, the size and shape of blade engagement arrangement 140 is selected so that blade engagement arrangements 140 can be inserted in the interior cavity C that is formed by the blades in the closed position. In operation, when the blades are in the closed position, second end portion 130 of blade alignment device 100 that includes blade engagement arrangement 140 is inserted into interior cavity C that is formed by the blades. Blade alignment device 100 is oriented such that each of retention structures 142 of blade engagement arrangement 140 at least partially surround a wall flange F1 on one blade and a wall flange F2 on an adjacently position blade as illustrated in FIG. 7. Once blade alignment device 100 is properly oriented relative to the blades, blade alignment device 100 is inserted further into interior cavity C until the front end of blade alignment device 100 is located at or near the front end of interior cavity C. Generally, the front end of blade alignment device 100 is inserted 50-100% (and all values and ranges therebetween) the longitudinal length of interior cavity C. The insertion of blade alignment device 100 into interior cavity C results in blade alignment device 100 retaining the position of the blades relative to one another while the blades are in the closed position. While blade alignment device 100 is inserted in interior cavity C, the blades of the retractor device can then be inserted in the patient until the blades are properly oriented in the patient. Thereafter, blade alignment device 100 can be removed from interior cavity C and the blades of retractor device 200 can thereafter by moved to the open position.

Blade alignment device 100 is therefore used to retain the position of the blades relative to one another while the blades are in the closed position, and thereby inhibit or prevent 1) the distal end of the blades of the retractor device from becoming misaligned due to contact with bone and/or tissue as the blades of the retractor system are inserted into the patient, 2) damage to the blades of the retractor device and/or other components of the retractor device due to one or more bent blades, the twisting of one or more blades, damage to the blade opening and closing mechanism, etc., 3) improper insertion of the retractor system into the patient, and/or 4) undesired injury or damage to bone and/or tissue as the blades of the retractor system are inserted into the patient.

These and other advantages will become apparent to those skilled in the art upon the reading and following of this description.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. A blade alignment device adapted for use with a retractor device comprising an elongated body and a blade engagement arrangement; said elongated body includes first and second ends, a first end portion, and a second end portion; said blade engagement arrangement located at said second end portion; said blade engagement arrangement including a first retention structure; said first retention structure configured to at least partially surround and/or engage a portion of a plurality of blades on the retractor device to facilitate in maintaining alignment and/or position of the plurality of blades relative to one another when the plurality blades are in a closed position.

2. The blade alignment device as defined in claim 1, wherein said elongated body has a generally cylindrical shape between said first end portion and said second end portion.

3. The blade alignment device as defined in claim 1, wherein said elongated body has an internal cavity that runs along 50-100% a longitudinal length of said elongated body.

4. The blade alignment device as defined in claim 1, further including a gripping member positioned on said first end portion of said elongated body; said gripping member including one or more features selected form the group consisting of a groove, a rib, a slot, and a roughened region; said gripping member is optionally located on no more than 30% of said longitudinal length of said elongated body.

5. The blade alignment device as defined in claim 1, wherein said first retention structure includes first and second side walls that are configured to at least partially entrap or surround at least a portion of a plurality of blades of the retractor device when the blades are in the closed position.

6. The blade alignment device as defined in claim 5, wherein said first and second side walls angle inwardly toward one another; said first and second walls optionally extending outwardly from an outer perimeter of said elongated body.

7. The blade alignment device as defined in claim 1, further including a second retention structure on said blade engagement arrangement; said second retention structure configured to at least partially surround and/or engage a portion of a plurality of blades on the retractor device to facilitate in maintaining alignment and/or position of the plurality of blades relative to one another when the plurality blades are in a closed position; said first and second retention structures are spaced from one another.

8. The blade alignment device as defined in claim 7, wherein said second retention structure includes first and second side walls configured to at least partially entrap or surround at least a portion of a plurality of blades of the retractor device when the blades are in the closed position; said first and second side walls on said second retention structure optionally angle inwardly toward one another; said first and second walls on said second retention structure optionally extending outwardly from an outer perimeter of said elongated body.

9. The blade alignment device as defined in claim 1, wherein at least one of said first and second retention structures have a generally C-shaped configuration.

10. The blade alignment device as defined in claim 1, wherein said blade engagement arrangement is spaced inwardly from said second end of said elongated body; said blade engagement arrangement is optionally spaced inwardly from said second end of said elongated body a distance of 0.5-20% said longitudinal length of said elongated body.

11. The blade alignment device as defined in claim 1, wherein said blade engagement arrangement has a longitudinal length that is less than said longitudinal length of said elongated body; said longitudinal length of said blade engagement arrangement is optionally 2-40% said longitudinal length of said elongated body.

12. A method for using a blade alignment device to at least partially maintain alignment and/or positioning of a plurality of retractor device blades relative to one another when said plurality of retractor device blades are in a closed position; said method comprising:
providing a retractor device; said retractor device comprising a body, first and second arms, a first blade connected to said first arm, and a second blade connected to said second arm; said first and second blades movable between a closed position and an open position; said first and second blades at least partially forming a blade cavity when said first and second blades are in said closed position;
providing a blade alignment device as defined in claim 1; said blade alignment device adapted for use with said retractor device;
at least partially inserting said blade alignment device in said blade cavity such that said first retention structure at least partially surrounds and/or engages a portion of said first and second blades to facilitate in maintaining alignment and/or position of said first and second blades relative to one another when said first and second blades are in said closed position.

13. The method as defined in claim 12, wherein each of said first and second blades includes first and second side walls that are optionally arcuate shaped, and wherein an end portion of each of said first and second side walls includes an outwardly extending wall flange; said first and second wall flanges run partially or fully along a longitudinal length of said first and second blades; and wherein said first retention structure is configured to at least partially surround, engage, and/or entrap a portion of said first side wall on said first blade and said second side wall of said second blade when said blade alignment device is at least partially inserted in said blade cavity.

14. The method as defined in claim 12, wherein said longitudinal length of said elongated body is 80-150% a longitudinal length of said first blade and/or said second blade.

15. A method for using a retractor device in a patient comprising:
providing a retractor device; said retractor device comprising a body, first and second arms, a first blade connected to said first arm, and a second blade connected to said second arm; said first and second blades movable between a closed position and an open position; said first and second blades at least partially forming a blade cavity when said first and second blades are in said closed position;
positioning said first and second blades of said retractor device in said closed position;
providing a blade alignment device as defined in claim 1; said blade alignment device adapted for use with said retractor device;
at least partially inserting said blade alignment device in said blade cavity such that said first retention structure at least partially surrounds and/or engages a portion of said first and second blades to facilitate in maintaining alignment and/or position of said first and second blades relative to one another when said first and second blades are in said closed position;
inserting at least a portion of said first and second blades of said retractor device into said patient until a front end of said first and second blades is located at a desired location in said patient;
removing said blade alignment device from said retractor device; and,
causing said first and second blades to move from said closed position to said open position while at least a portion of said first and second blades are located in the patient; and wherein said maintaining alignment and/or position of said first and second blades relative to one another by said blade alignment device optionally inhibits or prevents a) the distal end of the blades of the retractor device from becoming misaligned due to contact with bone and/or tissue as the blades of the retractor device are inserted into the patient, b) damage to the blades of the retractor device and/or other components of the retractor device due to one or more bent blades, the twisting of one or more blades, damage to the blade opening and closing mechanism, etc., c) improper insertion of the retractor device into the patient, and/or d) undesired injury or damage to bone and/or tissue as the blades of the retractor device are inserted into the patient.

16. The method as defined in claim 15, wherein each of said first and second blades includes first and second side walls that are optionally arcuate shaped, and wherein an end portion of each of said first and second side walls includes an outwardly extending wall flange; said first and second wall flanges run partially or fully along a longitudinal length of said first and second blades; and wherein said first retention structure is configured to at least partially surround, engage, and/or entrap a portion of said first side wall on said first blade and said second side wall of said second blade when said blade alignment device is at least partially inserted in said blade cavity.

17. The method as defined in claim 15, wherein said longitudinal length of said elongated body is 80-150% a longitudinal length of said first blade and/or said second blade.
